(19) United States
(12) Patent Application Publication
(10) Pub. No.: US 2009/0069835 A1
(43) Pub. Date: Mar. 12, 2009

(54) Balloon catheter for endoscopic mucosectomy

(76) Inventors: Massimo Conio, San Remo (IT); Marina De Matthaeis, Roma (IT)

Correspondence Address: Ralph A. Dowell of DOWELL & DOWELL P.C., 2111 Eisenhower Ave, Suite 406, Alexandria, VA, 22314, US

(21) Appl. No.: 11/898,281

(22) Filed: Sep. 11, 2007

(51) Int. Cl. A61B 1/32

(52) U.S. Cl. 606/190

(57) ABSTRACT

The balloon is fixed to a catheter close to its distal portion; the catheter has two ducts passing through within in; one duct can be used for a guide wire, while the other is used to pump air in or out; after passing through the catheter, the air emerges from a hole in the wall of the distal portion of the catheter, within the balloon; the distal portion of the catheter emerging from the balloon is rigid and slightly curved with respect to the axis of the catheter so that, when used, the distal portion—fitted with a sphere at the very tip—is curved upwards; the sides of the inflatable balloon are of varying thickness so that, when inflated, it takes on the form of a mattress; its inflation enables the detachment without trauma of two layers of tissue.

### FIELD OF APPLICATION

The present invention comprises of a balloon catheter to be used in endoscopy for the purposes of effecting a mucosectomy.

### STATE OF THE TECHNIQUE

Mucosectomy is an endoscopic technique in which areas of pathological mucosa are removed. This technique can be effected to remove fairly large lesions (up to 4 inches) from the digestive system, from the esophagus to the colon-rectum, to remove polyps and small superficial malignant tumours identified in an early diagnosis, when the illness is still at an initial phase.

The principle on which the mucosectomy is based is quite simple: the wall of hollow organs, such as the colon for example, is formed of four layers: the mucosa (the innermost stratum), the sub-mucosa, the muscle layer and the peritoneum. The exception being the esophagus, which has no peritoneum. At the moment, mucosectomies are effected by injecting a saline solution and vasoconstrictive drugs using a sclerosis needle to create a narrow hollow beneath the mucosa to be removed. The pathological mucosa is lifted and detached from the muscle layers beneath. The pathological mucosa is then removed using a diathermic snare.

Mucosectomy can be used to remove both benign and malignant lesions limited to the mucosa and the most superficial part of the sub-mucosa. It offers a number of benefits, the most important being the recovery of the resected lesion. This enables a careful histological examination and makes it possible to understand whether the operation has cured the pathology or whether other treatment be necessary. The operation is painless. It can be effected with or without anesthesiological assistance and the patient be discharged from hospital 24-48 hours after the endoscopic examination.

At the moment, only the liquids mentioned above are used to detach the mucosa from the underlying layers—injected using a sclerosis needle. However, this process runs the risk of causing a hemorrhage during the lifting and detachment of the mucosa, or of perforating the viscus during the detachment or resection of the lesion.

The endoscopic sector is already aware of small balloon catheters of various sizes and in both cylindrical and spherical form. Cyclindrical balloons are used to dilate some restricted sections of the digestive system, while spherical ones are generally used to remove calculi from the biliary tract. But when they are inflated, both spherical and cylindrical balloons exercise a radial pressure that is ideal for the above purposes, but not for detaching the mucosa from the sub-mucosa.

### SUMMARY OF THE INVENTION

The aim of the present invention is to provide a balloon catheter able to separate without trauma the two layers of tissue in the wall of the digestive apparatus. This balloon catheter consists of an inflatable balloon of flexible and resilient fixed close to the distal end of a catheter. The catheter is traversed internally by at least two ducts providing the means to inflate and deflate the balloon and by a guide wire. The distal end of the catheter emerging from the balloon is rigid, slightly curved and ends with a sphere in which there is a channel through which passes the guide wire; this emerges from the sphere via a hole. The inflatable balloon has a varying thickness so that, once inflated, it takes on the form of a mattress; that is, roughly a parallelepiped.

The mattress shape of the balloon makes it possible to exercise an opposing bi-directional pressure resulting in the uniform detachment of the two layers of tissue. While the balloon is inflated, the lifting of its broad, flat side causes the simultaneous lifting of a corresponding area of tissue, which is detached from that beneath. On the underside, a pressure on the underlying tissue is maintained with hemostasis of the injured vessels during the maneuver, thereby preventing any possible hemorrhage.

### BRIEF DESCRIPTION OF THE DRAWINGS

The invention will now be further described with reference to the accompanying drawings, in which:

FIG. 1 shows a perspective view of the whole of the balloon and catheter;

FIG. 2 shows a view from above of the balloon catheter when inflated and of the distal part of the catheter;

FIG. 3 shows a side view of the balloon catheter when inflated and of the distal part of the catheter;

FIG. 4 shows a frontal view of the balloon catheter when inflated and of the distal part of the catheter;

FIG. 5 shows a longitudinal cross-section of the inflated balloon catheter and of the distal part of the catheter;

FIG. 6 shows, in increased detail, part of the same longitudinal cross-section of the catheter as shown in FIG. 5.

### DESCRIPTION OF THE PREFERRED FORM

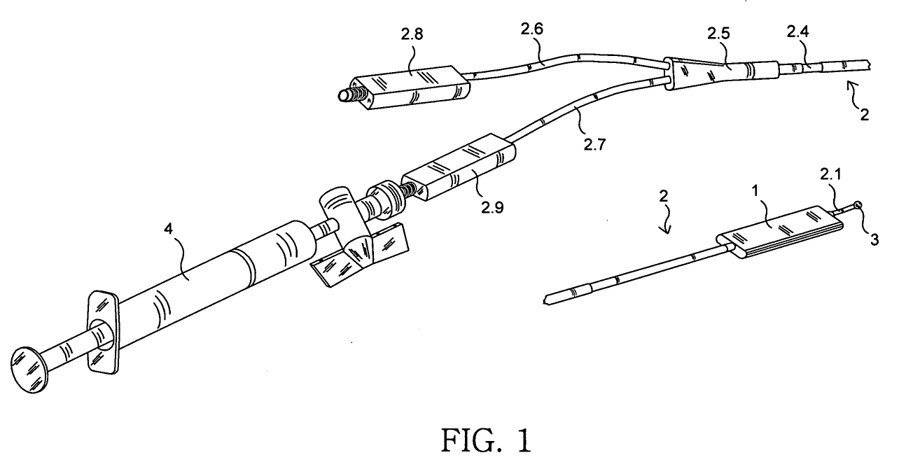

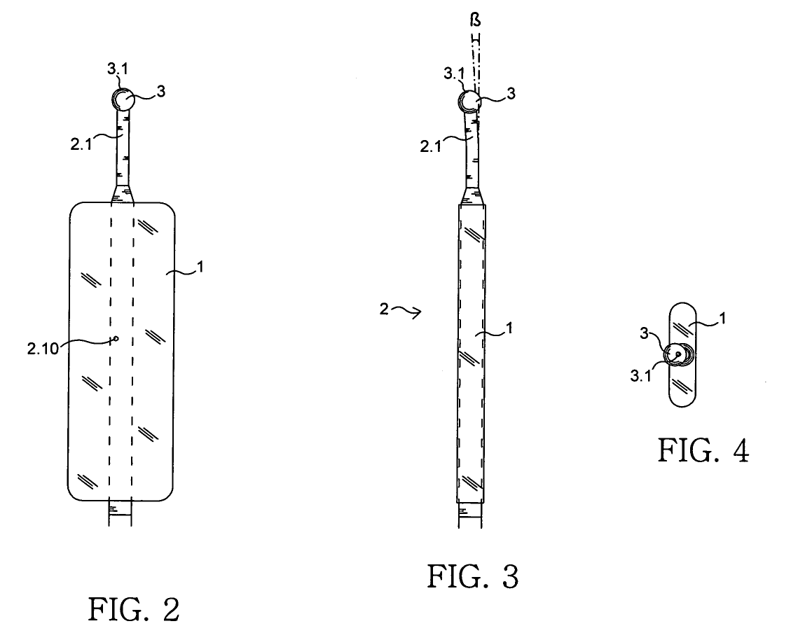

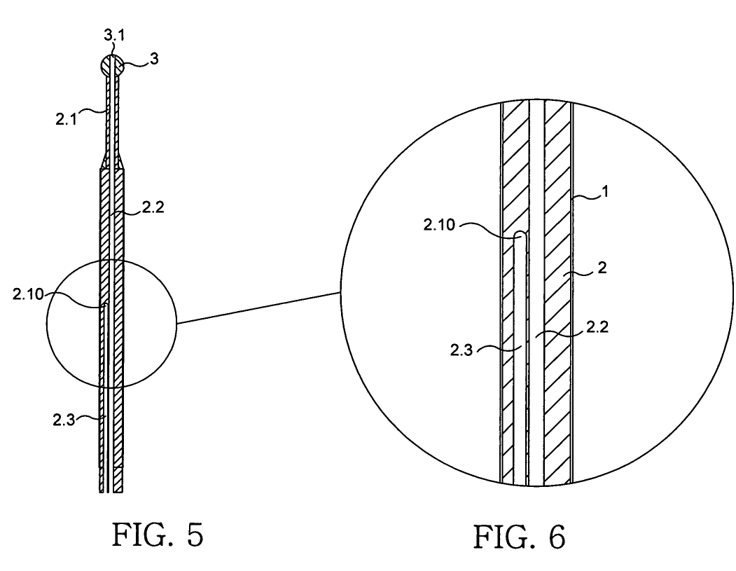

FIGS. 1 to 6 illustrate the balloon catheter of the present invention. Balloon 1 is a hollow tube formed of a flexible and resilient material. Its sides are of varying thickness so that, when inflated, it takes on the form of a mattress; that is, as flat as possible.

Balloon 1 is inserted on a catheter 2. The ends of the balloon 1 are fixed through fusion to catheter 2 near its distal end 2.1.

Catheter 2 has two ducts 2.2, 2.3 running through its interior. A joint 2.5 is fixed nearer in on the catheter 2 at proximal portion 2.4, to which the ends of two small tubes 2.6, 2.7 are attached. Joint 2.5 also has the two ducts in catheter 2 running through it, and these continue separately into each small tube 2.6, 2.7. At the other end of each tube 2.6, 2.7 are fixed connectors 2.8, 2.9, each with an externally threaded socket on the opposite end to that on which is fixed either tube 2.6 or 2.7. The ducts present in catheter 2, which continue separately in either tube 2.6, 2.7, pass through the connectors 2.8, 2.9 and carry on to the open end of the threaded sockets of the same connectors 2.8, 2.9.

The distal end 2.1 of the catheter 2, emerging from balloon 1, is rigid. It is slightly curved with respect to the axis of the catheter 2 at an angle β so that, when used, distal end 2.1 is curved upwards. This slight but important curvature of the point of the catheter 2 enables the safe arrival of the balloon 1 below the lesion and its exit via the normal mucosa, beyond the lesion to be removed, avoiding a situation whereby in being positioned below the lesion, the point downward and hence towards the deeper layers of the tissue with the consequent risk of perforating the viscus.

A sphere 3 is fixed to the distal end 2.1 of the catheter 2, through which passes a duct emerging on the far side via a hole 3.1. The sphere 3 constitutes the atraumatic point of the catheter 2.

A guide wire is threaded through connector 2.8 and passed through tube 2.6, joint 2.5, duct 2.2 and emerges from hole 3.1 in sphere 3.

The balloon 1 is inflated by injecting air into its interior via a syringe 4. The syringe is screwed on to connector 2.9 and the flow of air created by it passes through connector 2.9, tube 2.7, joint 2.5 and duct 2.3 in catheter 2, emerging from a hole 2.10 in the side of the distal end 2.1 of catheter 2, located within balloon 1.

In operational terms, on the initial movement effecting a minimal infiltration of the mucosa, the guide wire enters the tiny space and perfectly guides the introduction of catheter 2. At this point, the balloon is gradually inflated, obtaining a progressive detachment of the layer of mucosa located above it. At the same time, the layer beneath is compressed and the pressure exercised causes a hemostasis of any small vessel that might have been damaged during the maneuver.

1. A balloon catheter, which balloon is a hollow tube formed of a flexible and resilient material inserted on a catheter and fixed to it close to its distal end by fusion of the extremities of the balloon to the catheter, which catheter contains at least two ducts, one of which providing a passage through which passes the air to inflate or deflate the air for the balloon, the air passing through a hole on the wall of the distal end of the catheter within the balloon itself; said balloon is for use with a catheter for the purposes of endoscopic mucosectomy and makes it possible to apply bi-directional and opposing pressure, determining the uniform detachment of the two layers of tissue, wherein:
the sides of the inflatable balloon are of varying thickness to ensure that, once inflated, the balloon takes on the form of a mattress, that is approximately a parallelepiped;
the distal end of the catheter emerging from the balloon is rigid, slightly curved and ends in a sphere;
at least one duct within the catheter and one duct within the sphere provide the passage for a guide wire, which emerges from a hole set in the sphere.
